(19) United States
(12) Patent Application Publication
(10) Pub. No.: US 2005/0055041 A1
(43) Pub. Date: Mar. 10, 2005

(54) Device for separation of corneal epithelium

(75) Inventor: Stephen P. Woods, Hertfordshire (GB)

Correspondence Address: GARDNER GROFF, P.C.;PAPER MILL VILLAGE, BUILDING 23, 600 VILLAGE TRACE, SUITE 300, MARIETTA, GA, 30067, US

(73) Assignee: SIGHTRATE B.V., Amsterdam (NL)

(21) Appl. No.: 10/935,539

(22) Filed: Sep. 7, 2004

Related U.S. Application Data
(60) Provisional application No. 60/500,874, filed on Sep. 5, 2003.

(51) Int. Cl. A61F 9/00

(52) U.S. Cl. 606/166

(57) ABSTRACT

A drive tool for optical surgery includes a handpiece containing a traverse motor and an oscillating motor, wherein the oscillating motor is axially driven through the handpiece by the traverse motor. A head assembly including a suction ring is mounted to the handpiece, and a separator drive assembly is positioned therein for advancement imparted by the traverse motor and oscillation imparted by the oscillating motor.

### CROSS-REFERENCE TO RELATED APPLICATION

This application claims priority benefit to U.S. Provisional Patent Application Ser. No. 60/500,874, filed Sep. 5, 2003, the entirety of which is hereby incorporated by reference herein.

### TECHNICAL FIELD

The present invention relates generally to ocular surgical devices, and more particularly to a surgical apparatus and method for separating the epithelium layer of a cornea from the underlying Bowman's layer with minimal trauma to the epithelium and Bowman's layer.

### BACKGROUND OF THE INVENTION

Microkeratome devices are widely used in LASIK (Laser-Assisted In Situ Keratomilousis) procedures. LASIK permanently changes the shape of the cornea, the clear covering of the front of the eye, using an excimer laser. A microkeratome is used to cut a corneal flap, typically containing an overlying layer of corneal epithelium, Bowman's layer, and a portion of the stroma by slicing through the stroma, dividing it into at least two distinct portions. A hinge of uncut corneal tissue is typically left at one end of this flap. The flap is folded back revealing the penetrated stroma, the middle section of the cornea. Pulses from a computer-controlled laser vaporize a portion of the stroma and the flap is replaced. Known LASIK procedures typically require that the blade of the microkeratome be exceedingly sharp in order to produce consistent and reproducible flaps.

Recently, procedures have been developed for improved ocular procedures wherein the epithelial layer is separated from underlying corneal tissue, leaving Bowman's layer intact for corneal reshaping. See for example, International Patent Application Publication No. WO 2004/056295 A1, which is hereby incorporated herein by reference in its entirety. It has also recently been discovered that the separation of the corneal epithelium can be accomplished using a blunt polymeric separator rather than a sharp keratome blade. See for example, International Patent Application Publication No. WO 2004/052254 A1, which is hereby incorporated herein by reference in its entirety.

Previously known microkeratome devices have not proven fully satisfactory to many practitioners. For example, it has been found that many known microkeratome devices are complex and difficult to assemble and disassemble properly, potentially leading to difficulties in sterilization for reuse, interruptions in the surgical procedure, unduly adding to the cost of the devices, and increasing the incidence of device failure. It has also been found that many known microkeratome devices are bulky and unwieldy in use, potentially resulting in user fatigue and increasing the risk of errors during a procedure. Previously known microkeratome devices also have not been found to be well suited to the newly developed procedures for separation of corneal epithelium.

Thus it can be seen that needs exist for an improved microkeratome apparatus that is ergonomically configured for comfortable and effective use by a practitioner, that produces the desired manner of corneal separation, and that is simple to properly assemble and use. Needs also exist for an improved apparatus and method for separation of the corneal epithelium from underlying Bowman's layer. It is to the provision of methods and apparatus meeting these and other needs that the present invention is primarily directed.

### SUMMARY OF THE INVENTION

In example forms, the present invention is an improved drive tool for use in ocular surgery. In preferred applications, the tool is well suited to driving a blunt polymeric separator to separate the corneal epithelium from underlying Bowman's layer for subsequent corneal reshaping. In alternate embodiments, the device of the present invention may find application as a drive tool for standard sharp microkeratome blades, as in traditional LASIK procedures. The device of the present invention is preferably simple and elegant in design and construction, minimizing the necessary components and optimizing their assembly configuration, thereby resulting in a compact, ergonomic and easily manipulated surgical tool. In particularly preferred embodiments, the device is configured for comfortable one-hand operation by the practitioner. The device preferably also includes integral assembly interlocks, simplifying the proper assembly sequence and preventing improper assembly and disassembly, and preventing operation if the device is not fully and correctly assembled. The device is preferably configured for connection and use with standard suction and drive controllers that many practitioners will already have available, and with which practitioners are familiar and experienced in operating.

In one aspect, the present invention is a drive tool for optical surgery. The drive tool preferably includes a traverse motor for advancing a separator element along an axial path, and an oscillating motor for imparting lateral oscillation of the separator element across the axial path as it is advanced. The traverse motor and the oscillating motor are preferably coaxially aligned with one another.

In another aspect, the invention is a drive tool for optical surgery. The drive tool preferably includes an outer housing defining a central longitudinal axis extending lengthwise therethrough. The drive tool preferably also includes means for advancing a separator along a path and means for oscillating the separator. The means for advancing the separator and said means for oscillating the separator are preferably positioned along the central longitudinal axis of the outer housing.

In still another aspect, the invention is a drive tool for optical surgery. The drive tool preferably includes a housing having a traverse motor mounted in the housing adjacent a first end, and an oscillation motor mounted in the housing adjacent a second end. The housing preferably has an aspect ratio of between 3.5 and 10.

In still another aspect, the invention is a drive tool for optical surgery. The drive tool preferably includes a suction chamber having an outer rim surrounding an open bottom, an upper panel defining an opening, and at least one castellation projecting from the upper panel between the outer rim and the opening in the upper panel.

In another aspect, the invention is a drive tool for optical surgery. The drive tool preferably includes a handpiece having a housing and a coupling movable axially toward and away from a first end of the housing; a head assembly for connection to the first end of the handpiece; and a drive assembly for holding a separator for movement along the head assembly.

In another aspect, the invention is a separator drive assembly for a drive tool for optical surgery. The separator drive assembly preferably includes a receiver for engaging a separator, and a driveshaft extending from the receiver.

In another aspect, the invention is a separator for optical surgery. The separator preferably includes a leading edge and a rear face opposite the leading edge, and the rear face preferably defines an oscillation slot extending generally perpendicular to the leading edge.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a drive tool for optical surgery according to one example embodiment of the present invention.

FIG. 2a is a side cross-sectional view of the device shown in FIG. 1, and FIG. 2b is a bottom view of a suction ring portion thereof.

FIG. 3 is a detailed assembly view of a head unit portion of the device shown in FIG. 1.

FIGS. 4a and 4b show cross-sectional and lower perspective detailed views of the separator drive assembly and suction ring portions of the device shown in FIG. 1.

FIG. 5 is a perspective view of a driveshaft portion of the device shown in FIG. 1.

FIGS. 6a and 6b show detailed views of a shaft coupling portion of the device shown in FIG. 1.

FIGS. 7a-7d show detailed views of a separator portion of the device shown in FIG. 1.

FIG. 8 shows a cross-sectional detail of the device of the present invention in use.

FIG. 9 shows a cross-sectional view of a drive tool for optical surgery according to another example embodiment of the present invention.

FIGS. 10a and 10b show cross-sectional views of a drive tool for optical surgery according to still another example embodiment of the present invention.

FIG. 11 shows a cross-sectional view of a drive tool for optical surgery according to yet another example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment within the scope of the invention.

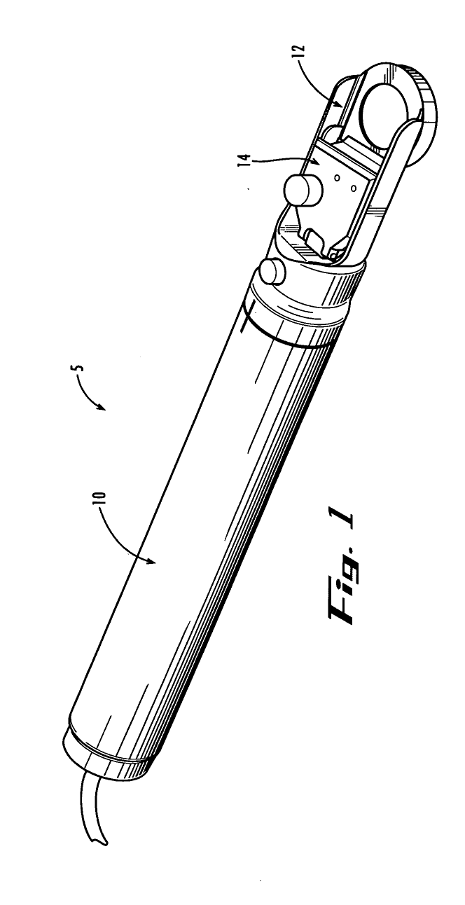

As seen best with reference to FIGS. 1-3, an example embodiment of the device 5 of the present invention generally comprises a handpiece 10, a head assembly 12, and a separator drive assembly 14. The handpiece 10 preferably comprises a generally cylindrical body having a circular cross-section of less than about one and one-half inches (1½″) in diameter, more preferably less than about one inch (1″) in diameter, and most preferably about ¾ inch (0.75″) diameter; and a length of less than about eight inches (8″), more preferably less than about six inches (6″), and most preferably about five inches (5″). These dimensions provide an aspect ratio (length/thickness) of between about 3.5 to about 10, and more preferably of about 5 to 7.5, which has been discovered to provide significant ergonomic advantage for carrying out the particular surgical procedures to which the device 10 is applicable. In particular, these dimensions, in combination with the weight and balance of the device 10, the electronic and suction coupling locations, and the manner of use, have been found to permit comfortable one-handed operation of the device by most practitioners for carrying out the intended procedures with a high degree of precision and minimal operator fatigue. Of course, it will be recognized that other configurations and dimensions are within the scope of the invention and may be advantageous for certain other applications. For example, alternate embodiments of the handpiece 10 are prismatic rather than cylindrical, for example having cross-sections that are triangular, square, hexagonal or other polygonal shapes, rather than having a circular cross-sectional geometry, along at least a portion of their length. While the depicted embodiment of the handpiece 10 comprises a substantially linear body having a generally constant cross-sectional geometry along its entire length, alternate embodiments incorporate a curved, stepped, or angled housing geometry, for example including one or more transverse or obliquely angled segments in the form of a pistol-grip or other configuration. The handpiece 10 preferably comprises a housing formed by an outer shell 16 and an inner shell 18, the inner shell configured to be slidably received within the outer shell with a close sliding fit. In example embodiments, the inner and outer shells, as well as the other structural components of the device 5, are formed of titanium, stainless steel and/or other substantially rigid, medical-grade material(s) suitable for autoclaving or other means of sterilization.

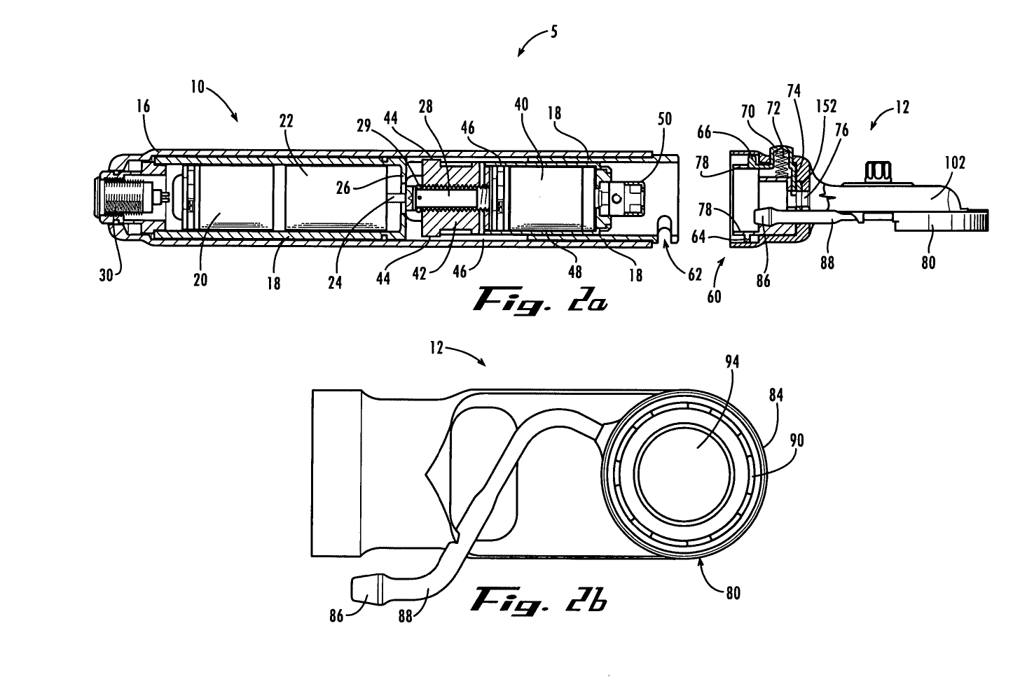

With reference to FIG. 2, a traverse motor 20 and gearbox 22 assembly is preferably fixedly mounted within a rearward bore formed in the distal end of the inner shell 18, as by screws 24 or other mounting means such as one or more rivets, adhesive, weldments, snap fittings or the like. In alternate embodiments, the gearbox 22 can be omitted by appropriate selection or control of the drive speed output of the traverse motor. An interior shoulder 26 is preferably formed in the bore of the inner shell 18 for securing the traverse motor 20 and gearbox 22 assembly in a fixed position relative to the inner shell. A drive screw 28 preferably extends forwardly from the gearbox 22, and is driven by the traverse motor 20 through the gearbox to advance and return the oscillating motor and separator drive assembly, as described in greater detail below. An O-ring or bushing 29 is preferably mounted at the rearward end of the drive screw 28 to limit the rearward travel of the oscillating motor. An electronic coupling 30 is mounted at the distal end of the housing of the handpiece 10, for connection to an external controller. The device 5 is preferably readily suited for use with standard commercially available external controllers, typically comprising electronic and suction outputs and foot-pedal input actuators. One or more cables, wires or other electrical conductors preferably extend in communication between a first set of terminals of the coupling 30 and the traverse motor 20. The motor 20 and, if present the gearbox 22, preferably operate to advance the separator at a rate of about 0.5 mm/second to about 6 mm/second, and more preferably at about 1 mm/second to about 4 mm/second. Suitable advance rates can be obtained, for example, utilizing a 12V, 1.2W electric motor having a maximum drive speed of 17,700 rpm, and a 67:1 reduction planetary gearbox.

An oscillating motor 40 is preferably translationally mounted to slide forward and rearward within the inner shell 18 of the housing of the handpiece 10 under the influence of the traverse motor 20, and to impart oscillatory motion to the separator. In an example embodiment, the oscillating motor 40 is identical to the traverse motor 20, for improved balance. In alternate embodiments, the oscillating motor 40 is a relatively high-speed motor, for example operating at up to about 100,000 rpm; and the traverse motor is a relatively low-speed motor, for example operating at about 15,000 rpm. The oscillating motor is preferably operated by an external controller to drive the separator at an oscillation rate of about 3,000 to about 20,000 cycles per second, and more preferably at about 5,000 to about 15,000 cycles per second. In alternate embodiments, one or more piezomechanical oscillators are utilized in place of the oscillating motor 40 to drive oscillatory motion of the separator. Wires or other electrical conductors preferably extend in communication between a second set of terminals of the coupling 30 and the oscillating motor 40 to provide power to drive the oscillating motor. The wires preferably include a loop or coil to provide sufficient slack to permit them to maintain electrical contact as the oscillating motor 40 advances and retracts, and the inner shell 18 preferably provides one or more guides or recesses for retaining the wires in place and preventing overextension or kinking of the wires as the oscillating motor advances and retracts. The oscillating motor 40 and the traverse motor 20 are preferably mounted in-line, coaxially within the body of the handpiece 10, to enable direct drive for both advancement and retraction of the separator, as well as lateral oscillation of the separator, and to permit a compact and ergonomic housing configuration. In alternate embodiments, the oscillating motor and the traverse motor are mounted in a side-by-side arrangement or with their axes laterally or angularly offset from one another. A bushing block 42 is preferably rigidly connected to the distal or rearward end of the oscillating motor 40, for engagement with the drive screw 28, and to constrain the travel of the oscillating motor to linear translation between a retracted position and an advanced position. In preferred form, the bushing block 42 is fabricated from polyetheretherketone (PEEK) or other medical grade engineering thermoplastic polymer(s). The bushing block 42 defines a generally central axial threaded bore, the threads mating with threads of the drive screw 28 such that the bushing block and attached oscillating motor 40 are advanced and retracted as the drive screw is rotationally driven by the traverse motor 20. Preferably, the drive screw 28 has threads only at its forward end, to minimize surface friction between the interengaging threads. One or more flanges 44 preferably project outwardly from the bushing block 42 (two flanges project from opposite sides of the bushing block in the depicted embodiment), and ride within slots 46 formed in the inner shell 18 to prevent the bushing block 42 and attached oscillating motor 40 from rotating within the inner shell, and optionally also to limit the forward and rearward travel of the bushing block and oscillating motor. The oscillating motor 40 and the bushing block 42 are preferably secured within a sleeve 48 that slides smoothly with a close fit within a forward bore formed in the forward end of the inner shell 18. By mounting the oscillating motor 40 toward the proximal end of the handpiece and the traverse motor 20 toward the distal end, the device has a balanced feel in the hand of the user, providing improved ergonomics.

The drive shaft of the oscillating motor 40 is preferably connected to a self-centering drive coupling 50, shown in greater detail in FIGS. 6a and 6b, for releasably engaging the separator drive assembly 14. The drive coupling 50 preferably comprises an opposed pair of springs 52, preferably formed of tubular segments of a resilient, self-damping polymeric material such as PEEK. The springs 52 compress inwardly against the driveshaft, as seen in FIG. 6a, to receive and release the coupling end of the shaft of the separator drive assembly 14, and then spring back in the opposite direction to engage and retain the shaft in the coupling 50 and to assist in maintaining the shaft centered in the coupling. An opposed pair of alignment fins 54 assist in maintaining the position and orientation of the springs and aligning the shaft concentrically within the coupling. The interaction of the springs 52 and the fins 54 with the shaft of the separator drive assembly in the coupling 50, serves to align and center the shaft and to provide a quick-release coupling for engaging and releasing the shaft as the device is assembled and disassembled.

The separator head assembly 12 preferably comprises a cylindrical distal end 60 for releasable connection to the handpiece 10. A bayonet coupling comprising an L-shaped slot 62 formed in the forward end of the inner shell 18 of the handpiece 10 cooperatively receives and engages an internal strut 64 in the distal end 60 of the separator head assembly 12 to secure the separator head assembly to the handpiece. The bayonet coupling is engaged by axially sliding the head assembly onto the handpiece, and then twisting the head assembly relative to the handpiece to engage strut 64 in the transverse portion of the slot 62. The distal end 60 of the separator head assembly 12 preferably further comprises a stop member 78 for contacting the forward end of the oscillating motor 40 to limit the forward travel of the oscillating motor and bushing block 42 assembly as it is advanced by the traverse motor during operation. In the depicted embodiment, the stop member 78 is an axially-extending, ring-shaped flange, but in alternate embodiments comprises one or more posts, fins or other limit member(s).

When the head assembly 12 is properly assembled on the handpiece 10, an internal spring-biased pin 66 in the distal end 60 of the head assembly engages within a hole 68 formed in the forward end of the inner shell 18 of the handpiece 10 to prevent inadvertent rotation and removal of the head assembly from the handpiece. A button 70 connected to the pin 66 allows the user to compress the spring 72 and retract the pin for assembly and disassembly. A finger 74 extends from the button 70 to at least partially block a passage 76 extending axially through the head assembly 12 when the button 70 is depressed. Abutment of the finger 74 against the shaft of the separator drive assembly 14, prevents the button 70 from being depressed when the shaft is installed through the passage 76, thereby preventing release of the pin 66 from the hole 68, and serving as a safety interlock to prevent detachment of the head assembly 12 from the handpiece 10 once the drive assembly has been installed. Also, the finger 74 preferably does not move clear of the passage 76 until attachment of the head assembly 12 to the handpiece 10 is complete and the pin 66 is fully engaged in the hole 68, thereby serving as a further safety interlock by preventing installation of the separator drive assembly 14 if the head assembly has been partially but incompletely installed. And if the user attempts to assemble the device by installing the drive assembly 14 prior to attaching the head assembly 12 onto the handpiece 10 (rather than the correct assembly sequence wherein the head assembly 12 is attached to the handpiece 10 before installing the drive assembly 14), the shaft of the drive assembly 14 will preferably prevent the button 70 from being depressed, and the pin 66 will not retract, thereby preventing the head assembly from being mounted onto the handpiece using an incorrect assembly sequence.

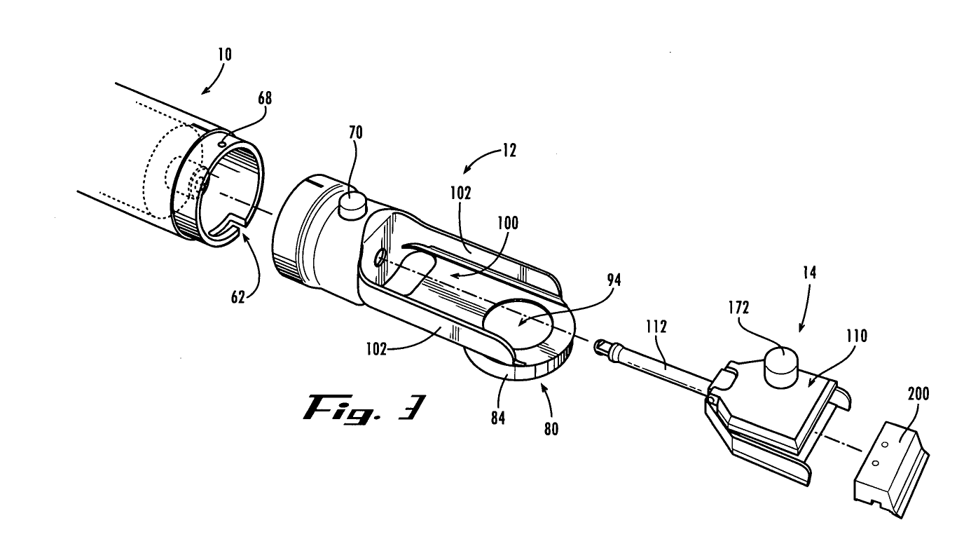

As seen best with reference to FIGS. 2b, 4a and 4b, a suction ring 80 is preferably provided at the forward end of the head assembly 12 for suction attachment to the eye being treated. The suction ring 80 preferably comprises a flat cylindrical chamber 82, open on the bottom for receiving the cornea of the eye into the chamber, and bounded by an outer circumferential flange 84. The lower edge of the outer circumferential flange 84 is preferably beveled or radiused to generally match the curvature of the eye and form an airtight seal between the flange and the eye. A suction coupling 86 is preferably provided for attachment to an external vacuum source, and a segment of tubing 88 extends in fluid communication between the suction coupling and the chamber 82 of the suction ring 80. A slotted inner flange 90 comprising a plurality of spaced castellations or fins 92 is preferably arranged within the chamber 82, spaced a distance inwardly from and generally concentric with the outer circumferential flange 84, to prevent tissue from obstructing fluid communication with the vacuum source, distribute the vacuum evenly around the suction ring 80, and thereby ensure a more secure suction attachment of the suction ring to the cornea. The walls of the chamber 82 preferably comprise one or more perforations or openings around or through one or more of the castellations, in fluid communication with the lumen of the tubing 88, for application of suction within the chamber. The castellations or fins 92 of the slotted inner flange 90 are preferably shorter than the height of the outer circumferential flange 84 and preferably are beveled or radiused on their distal or lower edges to generally match the curvature of the eye. A lower panel 91 is optionally provided, extending transversely inward from the outer circumferential flange 84, to define a plenum 93 bounded by the lower panel, the outer circumferential flange, the castellations and the top panel of the suction ring. The plenum 93 is in fluid communication with a suction delivery port through the outer circumferential flange 84 to deliver suction from the suction tube 88 via multiple ports formed between adjacent castellations, thereby providing more even application of suction and more consistent attachment to the cornea. The lower panel 91 preferably extends to a position just short of contact with the castellations 92, leaving a small gap therebetween to allow for some degree of suction delivery from the plenum 93 directly into the lower portion of the chamber 82. An upper opening 94 preferably extends through the top panel of the suction ring 80, generally concentric with the outer circumferential flange 84 and the slotted inner flange 90, through which upper opening 94 the cornea bulges upon suction attachment of the suction ring 80 to the eye. The inner rim of the upper opening 94 is also preferably beveled or radiused to generally match the curvature of the eye. The diameter of the opening 94 is preferably between about 8 mm to about 13 mm, and more preferably between about 10 mm to about 12 mm, for carrying out procedures on adult human eyes of average size. Of course, in alternate embodiments, the diameter of the opening 94 can be smaller or larger depending on the intended patient and application. The relative heights and spacing of the outer circumferential flange 84 and the castellations 92 permit their distal ends to engage the surface of the treated eye as the suction ring 80 is attached to the eye by application of suction, causing a portion of the cornea to be separated to bulge through the opening 94.

The head assembly 12 preferably further defines a guide channel 100, between the upper opening 94 of the suction ring 80 and the passage 76, for guiding the travel of the separator drive assembly 14. The guide channel 100 is preferably bounded on each side by a sidewall 102. The path of the guide channel 100 directs the leading edge of the separator 200 across the upper opening 94 as the drive assembly is advanced under the influence of the traverse motor 20.

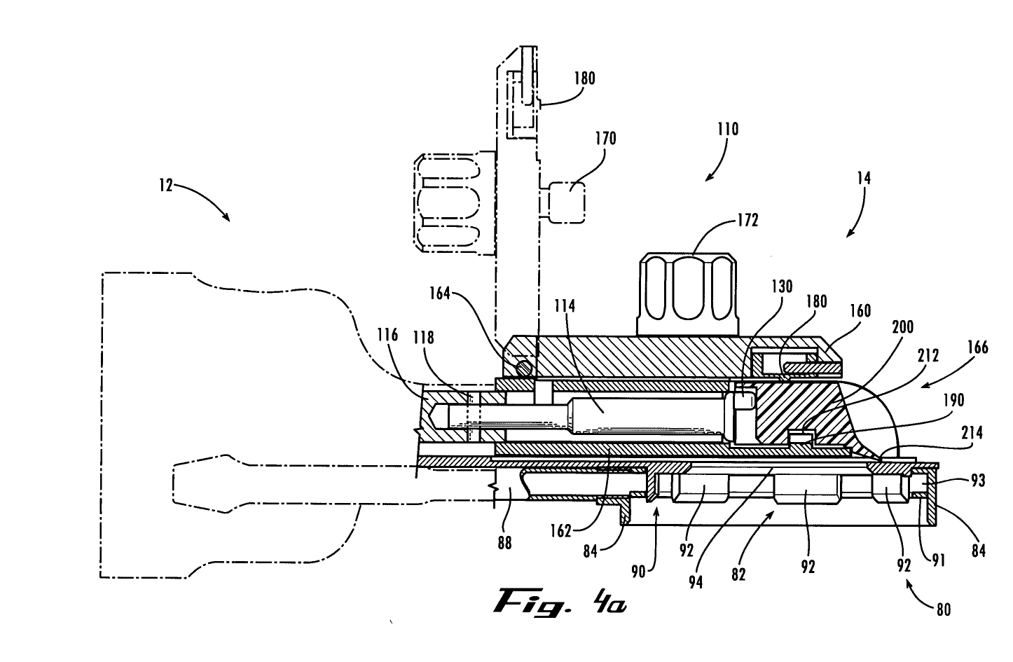

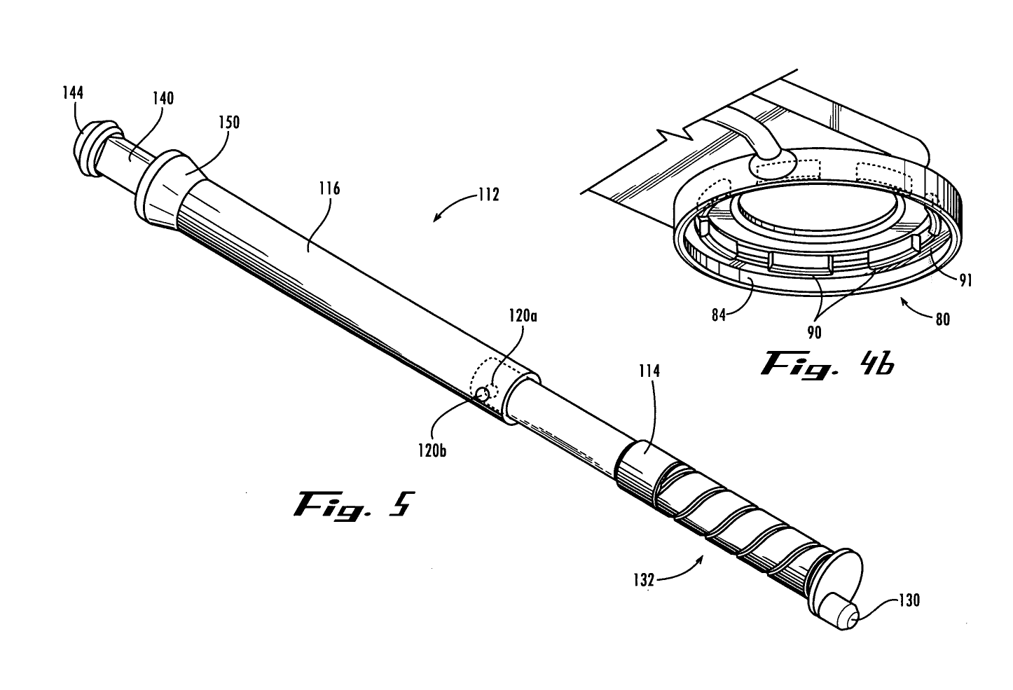

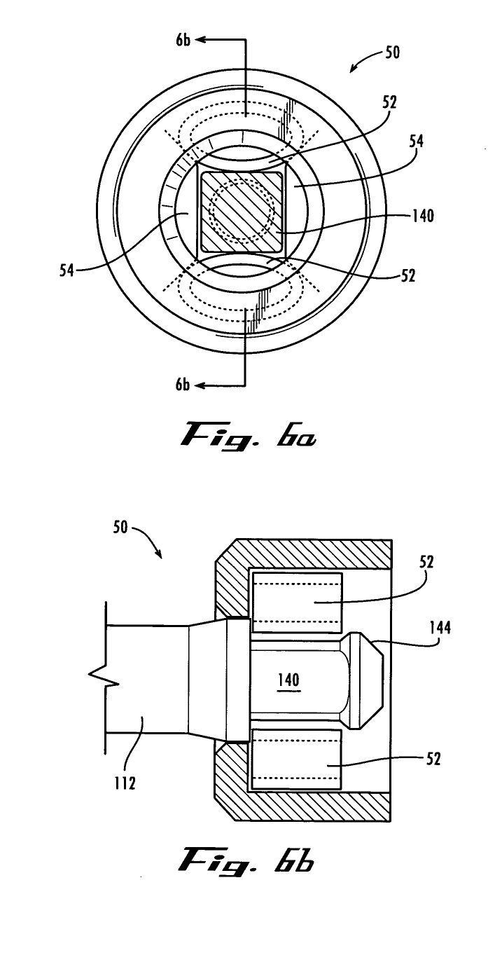

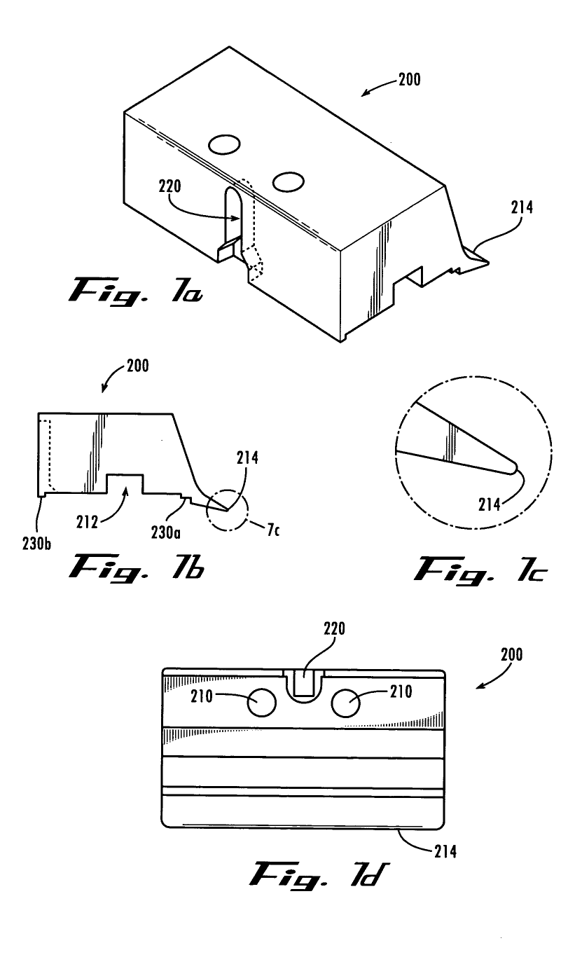

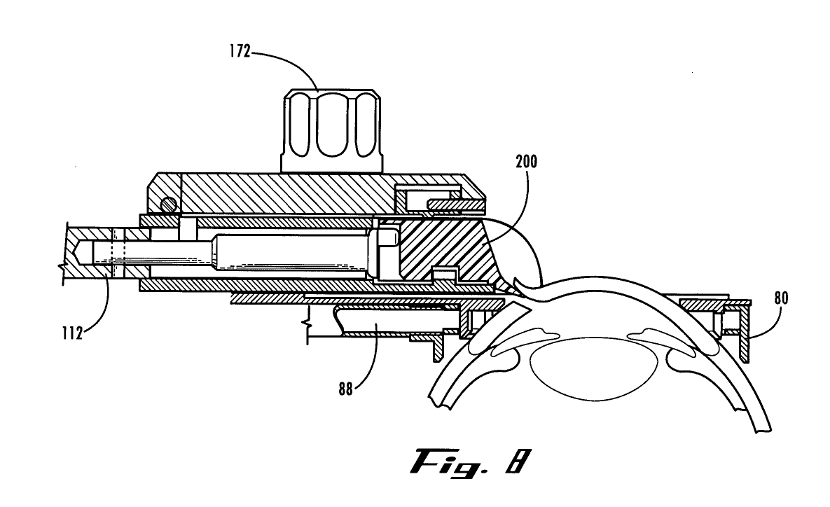

With reference now to FIGS. 3-5, the separator drive assembly 14 preferably comprises a separator coupling 110 and a driveshaft 112. The driveshaft 112 preferably extends through a bore in the separator coupling 110. In preferred form, the driveshaft 112 is a two-part shaft comprising a forward shaft segment 114 and a rear shaft segment 116 (see FIG. 5). In alternate embodiments, the driveshaft comprises a unitary component. The two-part driveshaft 112 is preferably assembled by inserting the forward and rear shaft segments into the bore of the separator coupling from opposite ends, and then connecting the overlapping ends of the shaft segments by press-fitting a pin 118 through cooperating holes 120a, 120b in the shaft segments. The forward shaft segment 114 preferably comprises an oscillation cam 130 extending axially from its forward end and laterally offset from the shaft's central longitudinal axis. The free end of the oscillation cam is preferably beveled or rounded to facilitate alignment and mounting of a separator head within the drive assembly. Grooves or vanes, for example in the form of helical reverse threading 132 are preferably provided along at least a portion of the forward shaft segment 114, to impel any liquid or debris out of the bore in the separator coupling 110 during use. The distal or rearward end of the rear shaft segment 116 preferably comprises a square drive segment 140 with a tapered tip or endcap portion 144 for engagement with the coupling 50 of the handpiece 10. The endcap portion 144 is preferably tapered on both its forward and rearward faces, to facilitate insertion into and removal from the coupling 50, and the square drive segment 140 is preferably configured to provide a close fitting engagement with the springs 52 of the coupling 50, as seen best with reference to FIGS. 5 and 6. The rear shaft segment 116 preferably further comprises a flared segment 150, tapered inwardly toward the forward end, to form an interference fit with a silicone washer 152 or other resilient member within the passage 76 through head assembly 12 to retain the separator drive assembly 14 in connection with the head assembly during insertion and removal, even when the square drive segment 140 is not positively engaged in the coupling 50, to prevent inadvertent displacement of the drive assembly. Optionally, a thrust tube is provided around the drive shaft 112 to bear the axial load during advancement, so as not to affect the rotary motion of the driveshaft, and to reduce or eliminate any variation in rotary speed or motor drive noise.

The separator coupling 110 preferably comprises an upper jaw member 160 that is hingedly connected to a lower jaw member 162 by a hinged pin joint 164. The separator coupling 110 defines a receiver opening 166 between the upper jaw member 160 and the lower jaw member 162 for receiving and engaging a separator 200. The receiver opening 166 is exposed for loading a separator 200 by opening the separator coupling 110 by pivoting the upper jaw member 160 away from the lower jaw member 162, as shown in broken lines in FIG. 4. After the separator 200 is loaded into the receiver opening 166, the upper jaw member 160 is pivoted closed over the lower jaw member 162, as shown in solid lines in FIG. 4, and a lockscrew 170 extending through the upper jaw member is tightened into a cooperating threaded opening in the lower jaw member 162 to lock the separator coupling 110 in its closed configuration and prevent removal of the separator 200. A knurled thumbwheel 172 is preferably affixed to the lockscrew 170 to assist in manually tightening the lockscrew. The inner face of the upper jaw member 160 preferably comprises a pair of flexible friction pads or lugs 180 projecting outwardly therefrom for contacting the upper face of the separator 200 and retaining it in position as it oscillates. The friction pads or lugs 180 are preferably fabricated from PEEK or other low-friction engineering polymer. The inner face of the lower jaw member 162 preferably defines a bearing surface upon which the separator 200 oscillates when in operation. An upright rib 190 preferably projects upwardly and extends laterally across the bearing surface, for cooperative engagement with a channel 212 in the lower face of the separator 200. The interengaging rib 190 and channel 212 maintain alignment of the separator as it oscillates, and also serve to prevent the separator coupling 110 from being closed if the separator 200 is installed in an improper orientation, serving as an additional safety interlock against improper or incomplete installation. Optionally, the separator 200 comprises one or more (two are shown) holes or recesses 210 for receiving cooperating engagement features of a gripping tool for assisting with insertion and removal of the separator into the separator coupling 110. The rear face of the separator 200 preferably defines a slot 220 for receiving the oscillation cam 130 of the driveshaft 112. The base of the slot 220 preferably tapers outwardly to facilitate engagement and alignment with the oscillation cam 130 upon assembly. The base of the separator preferably comprises relatively narrow forward and rear bearing pads 230a, 230b for riding on the bearing surface of the lower jaw member 162 of the separator coupling 110 to reduce friction, provide alignment and reduce the potential for seizing during oscillation in a fluid environment during use.

The separator 200 is preferably a disposable, single-use blunt separator of the type described in U.S. Provisional Patent Application Ser. No. 60/432,305, filed Dec. 10, 2002, which application is incorporated herein by reference. At least the leading edge 214 of the separator 200 is preferably formed of a plastic or polymeric material such as PEEK, PMMA, acetal homopolymer, polystyrene, MABS, and/or polycarbonate. The material of the separator 200 preferably will not withstand autoclave sterilization, thereby discouraging attempts to re-use a potentially contaminated separator. In alternate embodiments, the separator 200, including its leading edge 214, is formed of stainless steel or other sterilizable material(s) of construction. The separator drive assembly 14 is preferably a reusable assembly formed of stainless steel or other autoclavable material. In alternate embodiments, the entire separator drive and separator assembly are formed of plastics for economical disposability. The leading edge 214 of the separator 200 preferably has a radius of about 0.015 mm to about 0.025 mm, and is not sufficiently sharp to sever Bowman's layer of a typical human cornea, but rather, acts to separate the corneal epithelium from Bowman's layer as it is advanced through the cornea, leaving Bowman's layer intact. In alternate embodiments, the leading edge 214 of the separator 200 is sufficiently sharp to cut through the cornea.

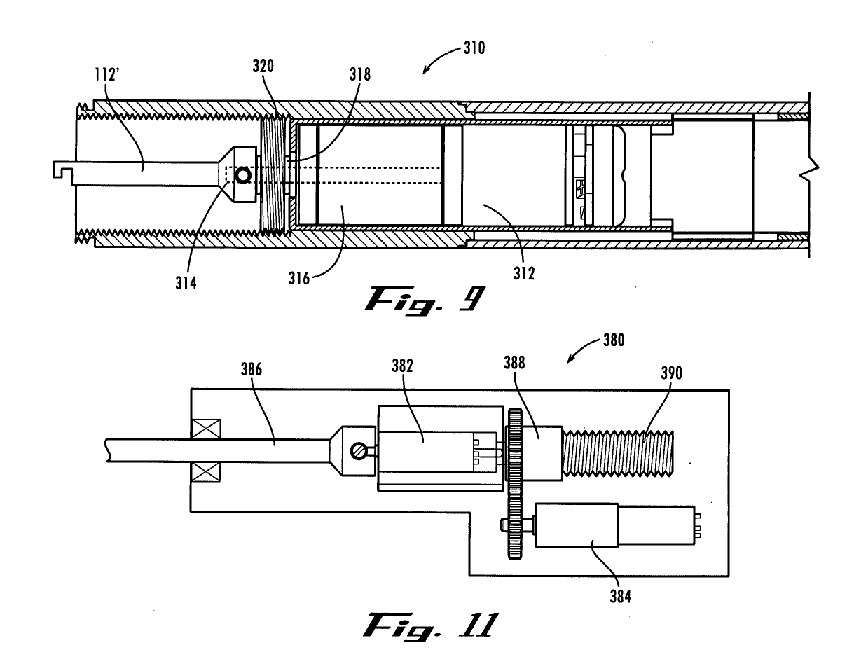

FIGS. 9-11 depict several alternate embodiments of a separator handpiece according to the present invention. In the handpiece 310 of FIG. 9, a single motor 312 serves to advance the separator drive assembly and to oscillate the separator. The drive shaft 314 of the motor 312 passes straight through an annular gearbox 316, and is connected to the driveshaft 112′ of the separator drive assembly to provide a high-RPM drive speed for imparting side-to-side oscillation to the separator. The annular gearbox 316 is driven by the drive shaft 314 of the motor 312, reduces the drive speed through gear reduction, and delivers a low-RPM drive via a hollow driveshaft 318 to turn a lead screw 320 within a threaded bore 322 to axially advance the separator drive assembly. Alternatively, a single high-speed drive motor having a driveshaft extending from each end can be utilized to both oscillate the separator and to advance the separator drive assembly. The front drive shaft couples to the driveshaft of the separator drive assembly to oscillate the separator, and the back drive shaft is coupled to a planetary gear with a controllable slip clutch mechanism. In this manner, the planetary output velocity is dependent on the clutch slip, and a fast closed-loop controller and slow-pitch lead screw are used to control the rate of advance.

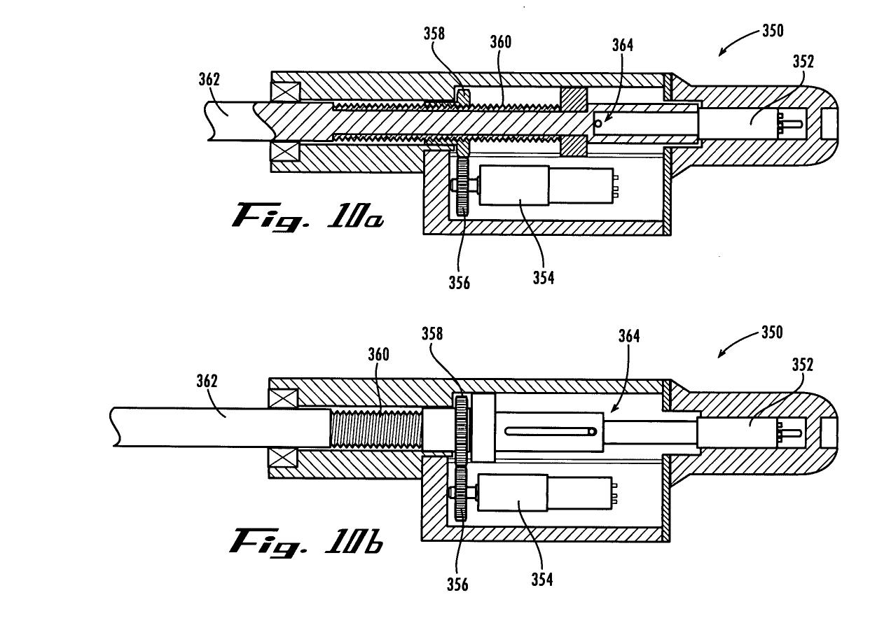

FIGS. 10a and 10b show a handpiece 350 having the oscillation motor 352 and the traverse motor 354 laterally offset a distance from one another, with their driveshaft axes generally parallel to one another. The oscillating motor 352 drives the driveshaft of the separator drive assembly at a high-RPM to oscillate the separator. A spur gear 356 coupled to the output of the traverse motor 354 drives an internally threaded journal gear collar 358 in engagement with an externally threaded transmission screw portion 360 of the driveshaft 362 to axially advance the driveshaft 362 and thereby advance the separator drive assembly. A pin-and-slot slip-coupling 364 in the driveshaft 362 allows extension (FIG. 10a) and retraction (FIG. 10b) of the driveshaft while transmitting rotational drive from the oscillation motor. FIG. 11 shows another handpiece 380 having a laterally offset configuration of the oscillation motor 382 and the traverse motor 384, wherein the oscillation motor and the driveshaft 386 are advanced and retracted by the traverse motor via an internally threaded journal gear collar 388 driven in engagement with an externally threaded transmission screw 390 to which the oscillation motor is mounted.

The device 5 is preferably assembled for use by sliding the head assembly 12 onto the handpiece 10 and twisting the head assembly to engage the bayonet coupling 62, 64 and lock the pin coupling 66, 68. A sterile separator 200 is loaded into the receiver opening 166 of the separator coupling 110, and the lockscrew 170 is tightened. The separator drive assembly 14 is then installed into the head assembly 12 by inserting the tapered endcap portion 144 of the driveshaft 112 through the passage 76 in the head assembly, and into engagement with the drive coupling 50 of the oscillating motor 40. An external vacuum source is connected to the suction coupling 86, and electrical leads from the external control device are connected to the electrical coupling 30.

In an example method of use, the suction ring 80 is affixed to the eye to be treated by application of vacuum, causing the cornea of the eye to bulge through the upper opening 94 of the suction ring. Typically, the controller will include foot pedal actuators for the vacuum source and the power to drive the motors, in order to allow the practitioner's hands to remain free for positioning and controlling the device 5 on the subject's eye. The oscillating motor 40 is actuated to rotationally drive the driveshaft 112, causing the offset oscillation cam 130 engaged within the slot 220 of the separator 200 to drive the separator in a laterally oscillating manner. The traverse motor 20 is actuated to drive the drive screw 28, which engages the threaded bore of the bushing block 42, driving the bushing block and oscillating motor assembly axially forward through the bore of the inner shell 18. The forward travel of the bushing block and oscillating motor assembly, in turn, drives the separator drive assembly 14 forward, causing the oscillating leading edge 214 of the separator 200 to move along the guide channel 100 and across the upper opening 94 of the suction ring, separating the corneal epithelium from the underlying Bowman's layer of the cornea, but preferably leaving Bowman's layer intact.

As the leading edge 214 of the separator approaches the forward extremity of the upper opening 94 of the suction ring, the stop member 78 abuts the forward end of the oscillating motor 40 to stop the forward travel of the oscillating motor and bushing block assembly, and thereby stop the forward advance of the separator drive assembly 14. Preferably, the travel of the separator drive assembly 14 is automatically stopped before the leading edge 214 of the separator reaches the forward extremity of the upper opening 94 of the suction ring, preventing complete detachment of the separated corneal epithelium and producing an epithelial flap, which remains attached to the eye at one end for replacement over the Bowman's layer after laser reshaping of the cornea. The traverse motor preferably stops upon abutment of the forward end of the oscillating motor against the stop member (the drive motor and gearbox are sized and configured to produce insufficient torque to strip the threads of the drive screw 28), signaling the controller that the forward travel is complete. The controller then de-activates the oscillating motor 40 and drives the traverse motor in the reverse direction to retract the oscillating motor and bushing block assembly, as well as the separator drive assembly coupled thereto. The application of suction ceases, and the device is removed from the eye. The exposed cornea may then be reshaped, as for example by excimer laser, and the epithelial flap replaced over the cornea for healing. Advantageously, the assembly and operation of the device of the present invention is the same for the left and the right eyes.

Finally, an applanator is included in some alternative embodiments. The applanator can take on many forms and can be composed of a variety of materials, and precedes the leading edge. In further embodiments, an applanator follows the leading edge; and in yet further embodiments, an applanator both precedes and follows the leading edge.

While the invention has been described with reference to preferred and example embodiments, it will be understood by those skilled in the art that a variety of modifications, additions and deletions are within the scope of the invention, as defined by the following claims.

1. A drive tool for optical surgery, said drive tool comprising a traverse motor for advancing a separator element along an axial path, and an oscillating motor for imparting lateral oscillation of the separator element across the axial path as it is advanced, wherein the traverse motor and the oscillating motor are coaxially aligned.

2. The drive tool of claim 1, wherein the traverse motor and the oscillating motor are mounted in counterbalancing positions within a housing, with the traverse motor adjacent a first end of the housing and the oscillating motor adjacent a second end of the housing.

3. The drive tool of claim 2, wherein the housing is cylindrical, having a central longitudinal axis that is generally coaxial with driveshaft portions of the traverse motor and the oscillating motor.

4. The drive tool of claim 2, wherein the housing has an aspect ratio of between about 5 to about 7.5.

5. The drive tool of claim 1, wherein the traverse motor rotationally drives a drive screw in threaded engagement with a bushing block coupled to the oscillating motor, to advance the oscillating motor.

6. The drive tool of claim 5, wherein the bushing block defines an internally threaded bore, and wherein the drive screw is threaded at a distal end only.

7. The drive tool of claim 1, further comprising a gearbox coupled to the output of the traverse motor.

8. The drive tool of claim 1, further comprising a coupling for releasably engaging a drive assembly that engages the separator element.

9. The drive tool of claim 8, wherein the coupling comprises an opposed pair of resilient springs, said springs releasably engaging a driveshaft portion of the drive assembly therebetween.

10. The drive tool of claim 9, wherein the coupling further comprising a pair of alignment fins for maintaining the driveshaft portion of the drive assembly centered between the opposed pair of resilient springs.

11. The drive tool of claim 1, wherein the traverse motor and the oscillating motor are mounted within a handpiece, said drive tool further comprising a head assembly for attachment to the handpiece, the head assembly defining a guide channel through which the separator element is advanced along its axial path.

12. The drive tool of claim 11, wherein the head assembly and the handpiece are connected by a bayonet coupling.

13. The drive tool of claim 11, further comprising a drive assembly for holding the separator element, said drive assembly comprising a driveshaft for insertion through a passage in the head assembly and connection to a releasable coupling of the handpiece.

14. The drive tool of claim 13, further comprising an interlock preventing insertion of the driveshaft through the passage unless the head assembly is fully engaged with the handpiece.

15. The drive tool of claim 13, further comprising an interlock preventing removal of the head assembly from the handpiece when the driveshaft is inserted through the passage.

16. The drive tool of claim 11, wherein the head assembly comprises a suction ring having an outer circumferential flange, an upper opening, and a plurality of castellations positioned between the outer circumferential flange and the upper opening.

17. The drive tool of claim 1, further comprising an applanator.

18. A drive tool for optical surgery, said drive tool comprising an outer housing defining a central longitudinal axis extending lengthwise therethrough, said drive tool further comprising means for advancing a separator along a path and means for oscillating the separator, said means for advancing the separator and said means for oscillating the separator being positioned along the central longitudinal axis of the outer housing.

19. The drive tool of claim 18, wherein the means for advancing a separator along a path comprises a traverse motor, and wherein the means for oscillating the separator comprises an oscillation motor.

20. The drive tool of claim 18, wherein the means for advancing a separator along a path and the means for oscillating the separator comprise a single drive motor.

21. The drive tool of claim 18, further comprising an applanator.

22. A drive tool for optical surgery, said drive tool comprising a housing having a traverse motor mounted in the housing adjacent a first end, and an oscillation motor mounted in the housing adjacent a second end, and wherein the housing has an aspect ratio of between 3.5 and 10.

23. The drive tool of claim 22, wherein the housing has a generally constant cross-sectional geometry along its length.

24. The drive tool of claim 22, wherein the housing is generally cylindrical.

25. The drive tool of claim 22, wherein the housing has an aspect ratio of between about 5 to about 7.5.

26. The drive tool of claim 22, wherein the housing has a thickness of less than about one and one-half inches.

27. The drive tool of claim 22, wherein the housing has a thickness of less than about one inch.

28. The drive tool of claim 22, wherein the housing has a length of less than about eight inches.

29. The drive tool of claim 22, wherein the housing has a length of less than about six inches.

30. The drive tool of claim 22, further comprising an applanator.

31. A drive tool for optical surgery, said drive tool comprising a suction chamber having an outer rim surrounding an open bottom, an upper panel defining an opening, and at least one castellation projecting from the upper panel between the outer rim and the opening in the upper panel.

32. The drive tool of claim 31, comprising a plurality of castellations projecting from the upper panel, arranged in a ring between the outer rim and the opening in the upper panel.

33. The drive tool of claim 32, further comprising a plenum within said suction chamber for delivery of suction through ports defined between adjacent castellations.

34. The drive tool of claim 31, wherein the outer rim comprises an angled distal face.

35. The drive tool of claim 31, wherein each castellation has a shorter height than the outer rim and comprises an angled distal face.

36. The drive tool of claim 31, further comprising an applanator.

37. A drive tool for optical surgery, said drive tool comprising:
a handpiece comprising a housing and a coupling movable axially toward and away from a first end of the housing;
a head assembly for connection to the first end of the handpiece; and
a drive assembly for holding a separator for movement along the head assembly.

38. The drive tool of claim 37, wherein the handpiece comprises a traverse motor for advancing and retracting the coupling toward and away from the first end of the housing.

39. The drive tool of claim 37, comprising an oscillation motor for rotationally driving the coupling as it moves toward the first end of the housing.

40. The drive tool of claim 37, wherein the head assembly and the handpiece are connected by a bayonet coupling comprising an L-shaped slot in the handpiece and an internal strut in the head assembly.

41. The drive tool of claim 37, wherein the coupling comprises a releasable coupling for releasably engaging a driveshaft of the drive assembly.

42. The drive tool of claim 41, wherein the head assembly has an axial passage therethrough for receiving the driveshaft of the drive assembly.

43. The drive tool of claim 42, further comprising an interlock preventing insertion of the driveshaft through the axial passage of the head assembly unless the head assembly and the handpiece are fully engaged with one another.

44. The drive tool of claim 42, further comprising an interlock preventing uncoupling of the head assembly from the handpiece when the driveshaft is inserted through the passage.

45. The drive tool of claim 37, wherein the head assembly defines a guide channel through which the separator advances as it moves along the head assembly.

46. The drive tool of claim 37, wherein the head assembly comprises a suction ring having an outer circumferential flange, an upper opening, and a plurality of castellations positioned between the outer circumferential flange and the upper opening.

47. The drive tool of claim 46, wherein a leading edge of the separator passes partially across the upper opening of the suction ring as it moves along the head assembly.

48. The drive tool of claim 37, wherein the drive assembly comprises a lower jaw member and an upper jaw member hingedly connected thereto, and defining a receiver opening between the upper and lower jaw members for receiving and engaging the separator.

49. The drive tool of claim 48, wherein the drive assembly comprises a rib for engagement with a cooperating channel of the separator to prevent the hinged upper and lower jaw members from being closed if the separator is improperly positioned in the receiver opening.

50. A separator drive assembly for a drive tool for optical surgery, said separator drive assembly comprising a receiver for engaging a separator, and a driveshaft extending from the receiver.

51. The separator drive assembly of claim 50, wherein the receiver comprises an upper jaw member pivotally connected to a lower jaw member, and movable between an open position for receiving and releasing a separator into the receiver, and a closed position for retaining a separator in the receiver.

52. The separator drive assembly of claim 51, wherein the separator can oscillate within the receiver in the closed position.

53. The separator drive assembly of claim 52, wherein at least one of the upper and lower jaw members comprise a transverse rib for engagement within a cooperating channel in the separator.

54. The separator drive assembly of claim 52, wherein at least one of the upper and lower jaw members comprise at least one flexible friction pad for engagement against the separator in the closed position.

55. The separator drive assembly of claim 50, wherein the driveshaft comprises a forward shaft segment and a rear shaft segment.

56. The separator drive assembly of claim 50, wherein the driveshaft comprises an oscillation cam extending axially from a forward end of the driveshaft and being laterally offset from a central longitudinal axis of the driveshaft.

57. The separator drive assembly of claim 50, wherein the driveshaft comprises helical reverse threading along at least a portion of its length.

58. The separator drive assembly of claim 50, wherein the driveshaft comprises a square drive segment at its distal end.

59. A separator for optical surgery, the separator comprising a leading edge and a rear face opposite the leading edge, said rear face defining an oscillation slot extending generally perpendicular to the leading edge.

60. The separator of claim 59, wherein at least one end of the oscillation slot comprises an outwardly tapered portion.

61. The separator of claim 59, further comprising a lower face defining a channel extending generally parallel to the leading edge.

62. The separator of claim 59, further comprising at least one installation hole for engagement with a gripping tool.
